(19) United States
(12) Patent Application Publication
(10) Pub. No.: US 2010/0030035 A1
(43) Pub. Date: Feb. 4, 2010

(54) Fuzzy system for cardiovascular disease and stroke risk assessment

(75) Inventors: Lawrence Wing Chi Chan, Kowloon (HK); Iris Frances Forster Benzie, Kowloon (HK)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: The Hong Kong Polytechnic University, Hong Kong (HK)

(21) Appl. No.: 12/222,132

(22) Filed: Aug. 4, 2008

(51) Int. Cl. A61B 5/00; G06Q 50/00

(52) U.S. Cl. 600/301; 705/2

(57) ABSTRACT

The present invention relates to a fuzzy system that provides a measure of global cardiovascular risk based on the risk factors associated with cardiovascular disease and stroke. The fuzzy system is of multiple input single output (MISO) wherein the inputs are the values of risk factors and the output is the measure of global cardiovascular risk.

### BACKGROUND

Cardiovascular disease (CVD) is one of the leading cause of the death and serious illness. Much is known about lifestyle risk factors and the pathogenesis of CVD, however, there are still significant gaps in knowledge relating to certain groups. Numerous studies have been performed to overcome these significant gaps.

In 1948, the Framingham Heart Study performed a study to identify the common factors or characteristics that contribute to CVD by following its development over a long period of time in a large group of participants who had not yet developed overt symptoms of CVD or suffered a heart attack or stroke. The Framingham Heart Study continues to make important scientific contributions by enhancing its research capabilities and capitalizing on its inherent resources.

The Atherosclerosis Risk in Communities (ARIC) Study is a prospective study conducted in four U.S. communities to investigate the etiology and natural history of atherosclerosis, investigate the etiology of clinical atherosclerotic diseases, and measure variation in cardiovascular risk factors, medical care and disease by race, sex, place, and time.

The Second Manifestation of ARTerial Disease (SMART) study had as its aim to examine the relation between asymptomatic carotid artery stenosis and the risk of vascular events in patients with various clinical manifestations of arterial disease or presence of type II diabetes mellitus but without a history of cerebrovascular disease.

While informative, new risk factors to CVD are consistently being located and the earlier studies have not been able to include these new risk factors. Thus, a new study or system is needed to study the effect of newly emerging risk factors to CVD, and being able to accept future, unknown risk factors.

It is an object of the present invention to overcome the disadvantages and problems in the prior art.

### DESCRIPTION

The present invention relates to a fuzzy system that provides a global risk measure for cardiovascular risk and cerebrovascular disease, with particular but not exclusive reference to those with type II diabetes mellitus. The fuzzy system is of multiple input single output (MISO) wherein the inputs are the values of risk factors and the output is the global risk measure for cardiovascular and cerebrovascular diseases. Through the present system, an individual's global coronary heart disease and stroke risk can be revealed using fuzzy logic and defuzzification, employing a wide range of demographic, physiological, biochemical, and molecular markers associated with coronary heart disease risk. The model has particular value and relevance to type II diabetic patients because of their increased risk of coronary heart disease that, currently, cannot be assessed at the individual level. An advantage over the prior art is the use of fuzzy logic to model changes in an individual's global risk by modulation of selected modifiable risk factors so that therapy can be targeted, thus modulating factors that would most effectively lower global risk in that individual.

These and other features, aspects, and advantages of the apparatus and methods of the present invention will become better understood from the following description, appended claims, and accompanying drawings where:

FIG. 1 shows the cardiovascular risk fuzzy system of the present invention;

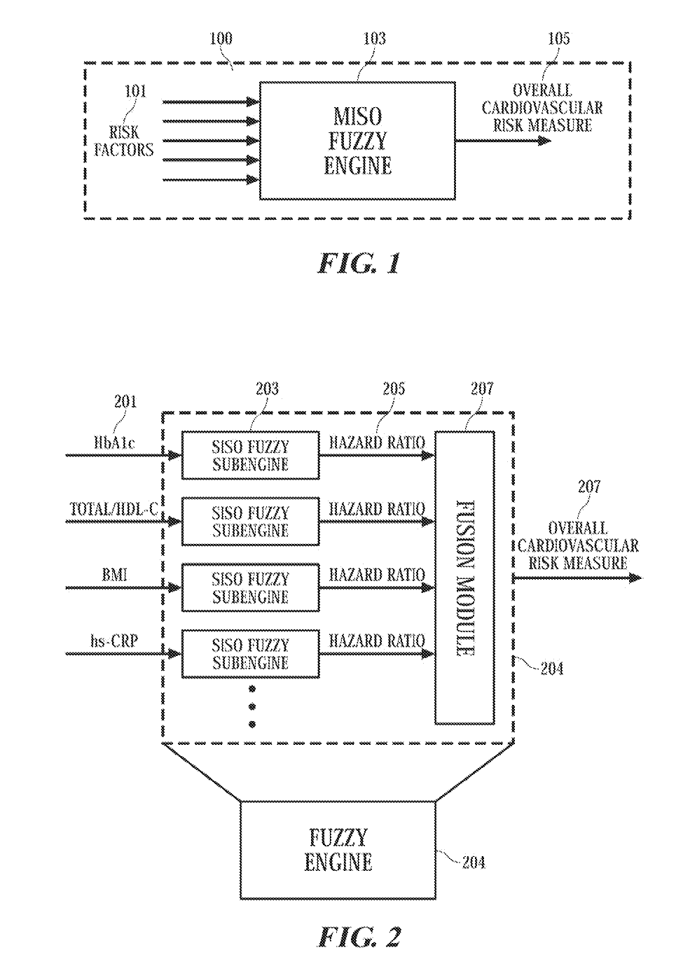

FIG. 2 exhibits the internal components of the fuzzy engine of the present invention;

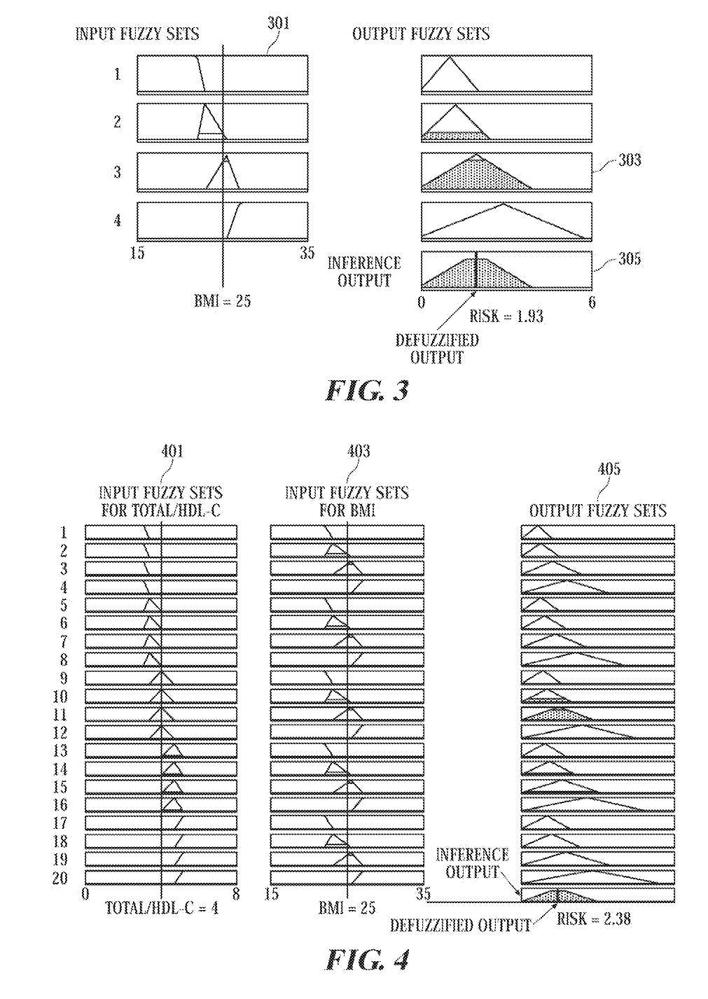

FIG. 3 shows the input/output of the fuzzy sets for the present system;

FIG. 4 is an embodiment of the system employing MISO subengine;

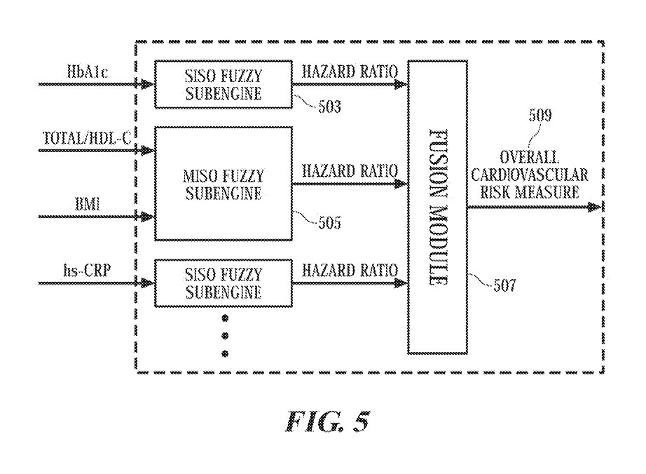

FIG. 5 exhibits the internal workings of a fuzzy engine containing SISO and MISO subengine.

The following description of certain exemplary embodiment(s) is merely exemplary in nature and is in no way intended to limit the invention, its application, or uses. Throughout this description, the term “values” shall refer to observed, recorded, measured, or elected data. The term “fuzzy” shall refer to numerical or non-numerical values whereby the boundaries of the value set is not fixed. The term “fuzzy function” shall refer to mathematical functions exhibiting fuzzy characteristics. The term “fuzzy engine” or “fuzzy subengine” shall refer to means for applying fuzzy functions to entered values; means can include process steps or algorithms.

Now, to FIGS. 1-5,

FIG. 1 is a cardiovascular risk fuzzy system 100 of the present invention.

The fuzzy system 100 includes risk factors 101 from a type II diabetes patient, serving as inputs into the engine 103. Risk factors 101 can be selected from age, smoking status, systolic blood pressure, cholesterol level, molecular, clinical and biomechanical measurements, gene expression, imaging examination findings, demographic data, family history, lifestyle, Coronary Heart Disease (CHD), stroke, and diet. Risk factors to be gauged can be one or more selected from the group consisting of gender, age, systolic blood pressure, diastolic blood pressure, body mass index (BMI), waist circumference, total cholesterol, low-density lipoprotein cholesterol, high density lipoprotein cholesterol, triglycerides, homocysteine, fasting glucose, type II diabetes (yes or no), duration of diabetes, serum or plasma, microalbuminuria (yes/no), history of vascular disease (yes or no), gene polymorphisms associated with CHD risk, diabetes mellitus, serum creatine, creatine clearance, smoking status (yes or no), history of smoking, medication use, vascular disease, fibrinogen, HbA1c, pulsatility index, spectral broadening index (SBI) of transcranial doppler ultrasound waveform, EDV ratio, carotid intima-media thickness (IMT), coronary artery calcium (CAC) score, flow-mediated endothelial vasodilatation, family history of CHD & stroke, and diet. The risk factors 101 can be expanded upon as new risk factors are discovered.

The fuzzy engine 103 accepts multiple risk factors 101 and delivers one measure, which is a global cardiovascular risk measure 105. As will be discussed later, the fuzzy engine possesses fuzzy algorithms.

The global cardiovascular risk measure 105 is the resultant from the fuzzy engine. The risk measure 105 is a reference to the cardiovascular risk of a type II diabetic patient. The risk measure 105 is the ratio of cardiovascular risk of a type II diabetic patient to that of the baseline.

FIG. 2 exhibits the internal components of the fuzzy engine in the present invention.

As shown and stated previously, risk factor 201 measurements or determinations are inserted into the fuzzy engine 204. The fuzzy engine 204 is comprised of separate fuzzy sub-engines 203 for each risk factor 201 input, separate hazard ratios 205 for each risk factor 201, and a fusion module 207 accepting risk factors 201 characterized by the hazard ratios 205.

The fuzzy sub-engines 203 represent the mapping of a particular risk factor to a corresponding hazard ratio 205. Each hazard ratio 205 maps a Universe of discourse for the corresponding risk factor 201 onto the Universe of discourse of output fuzzy sets; table 1 provides several examples of universes of discourse.

TABLE 1

| Universe | Set Range |
|---|---|
| Age | 0-100 years |
| Gender | Male/Female |
| Body Mass Index (BMI) | 18-35 |
| Diastolic Blood | 60-90 mmHg |
| Pressure | |
| Triglycerides | 1.69 mmol/L-5.65 mmol/L |
| Systolic blood | 110-140 mmHg |
| Pressure | |
| Smoker | yes/no |
| If not smoker | never or past smoker |
| Waist to Hip ratio | 0.7-0.9 |

The universes of discourse can be set by ranges as existing in a particular region or area.

A fuzzy inference is utilized to map the risk factor 203 within the fuzzy subengines 203. The inference can be selected from the well-known inferences in the art, such as Mamdani-style inference or Sugeno-style inference.

As known in the art, the last step in the inference is defuzzication. In the present invention, defuzzification occurs in the fusion module 207. Defuzzification can occur by the centroid technique, defined as the technique to locate the point where a vertical line would slice the fused outputs into two equal masses. The exponentially adjusted values of centroid of each output of hazard ratio are then multiplied with each other. The product of the multiplication is the global cardiovascular risk measure 207. The overall system 204 assumes that the risk factors 201 are independent of each other.

The fuzzy engine 204 can reside on a CPU of a computer system, and be represented by coded algorithms, such as
IF x is A
THEN y is B
   where x and y are variables, and A and B are values of the universes of discourses X and Y. U.S. Pat. No. 5,677,996, incorporated herein by reference, teaches a computer system useful in the present invention.

FIG. 3 exhibits rule evaluation applied to the inputted risk factors 301 and the output fuzzy sets 303, resulting in a defuzzifized output 305. These procedures are performed within the fuzzy engine, and subsequently the SISO fuzzy subengine, of the present engine.

In another embodiment of the present invention, two or more risk factors can be merged to form one multiple input single output (MISO) subengine prior to entering the fusion module. FIG. 4 exhibits the input of two risk factors, for example Total/HDL-C 401 and BMI 403, merged to deliver one output fuzzy sets 405. A defuzzified output 407 results after fusing all output fuzzy sets 405.

FIG. 5 exhibits a fuzzy engine 501 containing both single input single output subengines (SISO) 503 and multiple input single output subengines (MISO) 505. Both SISO 503 and MISO 505 are fed to the fusion module 507, which results in a global cardiovascular risk measure 509.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A fuzzy system for measuring cardiovascular disease and stroke risk with reference to type II diabetic patients, comprising
one or more risk factors; and
a fuzzy engine having single input single output fuzzy sub-engines equal in number to said risk factors whereby each risk factor is inserted into a corresponding sub-engine, a fusion module for accepting outputs from said subengines and delivering a global risk measurement.

2. The fuzzy system for measuring cardiovascular disease and stroke risk with reference to type II diabetic patients in claim 1, wherein said risk factors can be one or more selected from the group consisting of gender, age, systolic blood pressure, diastolic blood pressure, body mass index (BMI), waist circumference, total cholesterol, low-density lipoprotein cholesterol, high density lipoprotein cholesterol, triglycerides, homocysteine, fasting glucose, diabetes mellitus, serum creatine, creatine clearance, smoking status (yes or no), history of smoking, medication use, vascular disease, fibrinogen, HbA1c, APOE Gene polymorphism, pulsatility indes, spectral broading index (SBI) of transcranial doppler ultrasound waveform, EDV ratio, carotid intima-media thickness (IMT), coronary artery calcium (CAC) score, flow-mediated endothelial vasodilatation, family history of CHD & stroke, and diet.

3. The fuzzy system for measuring cardiovascular disease and stroke risk with reference to type II diabetic patients in claim 1, wherein said fuzzy sub-engine comprise fuzzy inferences that can be Mamdani-style or Sugeno-style.

4. A fuzzy system for measuring cardiovascular disease and stroke risk with reference to type II diabetic patients, comprising
one or more risk factors; and
a fuzzy engine having one or more single input single output fuzzy sub-engines, one or more multiple input single output fuzzy sub-engines, a fusion module for accepting outputs from said sub-engines, wherein said risk factors can correspond to a single input single output fuzzy sub-engine or two or more risk factors can correspond to the same multiple input output sub-engine.

5. The fuzzy system for measuring cardiovascular disease and stroke risk with reference to type II diabetic patients in claim 4, wherein said risk factors can be one or more selected from the group consisting of gender, age, systolic blood pressure, diastolic blood pressure, body mass index (BMI), waist circumference, total cholesterol, low-density lipoprotein cholesterol, high density lipoprotein cholesterol, triglycerides, homocysteine, fasting glucose, diabetes mellitus, serum creatine, creatine clearance, smoking status (yes or no), history of smoking, medication use, vascular disease, fibrinogen, HbA1c, APOE Gene polymorphism, pulsatility index, spectral broadening index (SBI) of transcranial doppler ultrasound waveform, EDV ratio, carotid intima-media thickness (IMT), coronary artery calcium (CAC) score, flow-mediated endothelial vasodilatation, family history of CHD & stroke, and diet.

6. The fuzzy system for measuring cardiovascular disease and stroke risk with reference to type II diabetic patients in claim 4, wherein said fuzzy sub-engines comprise fuzzy inferences that can be Mamdani-style or Sugeno-style.

7. A method of measuring cardiovascular disease and stroke risk in type II diabetic patients, comprising the steps of
obtaining one or more type II diabetic cardiovascular complication risk factors;
inserting said risk factors into an equal number or less of corresponding fuzzy sub-engines;
within said sub-engine, mapping said risk factors to an equal or less number of corresponding hazard ratios;
delivering mapped risk factors to fusion module;
fusing said risk factors via defuzzification within said fusion module; and
delivering a global cardiovascular risk measure.

8. The method of measuring cardiovascular disease and stroke risk in type II diabetic patients of claim 7, wherein obtaining said risk factors can occur by measurement, data entry, or observation.

9. The method of measuring cardiovascular disease and stroke risk in type II diabetic patients of claim 7, wherein each risk factor is inserted into a corresponding single input single output sub-engine, or two or more risk factors are inserted into one multiple input single output sub-engine.

10. The method of measuring cardiovascular disease and stroke risk in type II diabetic patients of claim 7, wherein mapping said risk factors occurs via fuzzy inference.

11. The method of measuring cardiovascular disease and stroke risk in type II diabetic patients of claim 7, wherein defuzzification occurs by the centroid of area technique.
